(19) United States
(12) Patent Application Publication
(10) Pub. No.: US 2025/0012800 A1
(43) Pub. Date: Jan. 9, 2025

(54) CALIBRATION SAMPLE FOR DETECTING ANTI-BARTONELLA HENSELAE ANTIBODIES

(71) Applicant: Medical Diagnostic Laboratories, L.L.C., Hamilton, NJ (US)

(72) Inventor: Annette DAUGHTRY, Hamilton, NJ (US)

(73) Assignee: Medical Diagnostic Laboratories, L.L.C., Hamilton, NJ (US)

(21) Appl. No.: 18/343,585

(22) Filed: Jun. 28, 2023

(51) Int. Cl. G01N 33/569

(52) U.S. Cl. CPC G01N 33/56983; G01N 2333/165; G01N 2469/20

(57) ABSTRACT

Provided is a method of preparing a calibration sample for detection of anti-Bartonella henselae antibodies in a subject, including obtaining one or more control sample, isolating antibodies therefrom, affinity purifying from the isolated antibodies that specifically bind target Bartonella henselae 17 kDa antigen or a fragment thereof to obtain a solution of purified antibodies, and diluting the solution of purified antibodies to a predetermined protein concentration to obtain the calibration sample. Also provided is a method for using the calibration sample for detecting anti-17 kDa antigen antibodies in a sample and treating a subject in whose sample anti-17 kDa antigen antibodies are detected, and a kit for performing the immunoassay including a calibration sample prepared as described.

### SEQUENCE LISTING

The instant application contains an electronic sequence listing. The contents of the electronic sequence listing H2704011.xml; Size: 12,564 bytes; and Date of Creation: Jun. 22, 2023, is herein incorporated by reference in its entirety.

### BACKGROUND

Bartonella henselae is a bacterium and the causative agent for Cat Scratch Disease in humans. Domestic cats are chronically infected with Bartonella henselae. They transmit the bacteria to humans through scratches or bites (Bergmans, et al., 1995). A variety of diagnostic assays exist but such assays require (i) culturing, (ii) immunofluorescence assay (“IFA”) and (iii) polymerase chain reaction (“PCR”) technologies. Culturing Bartonella henselae from blood samples is a difficult procedure which often yields low results.

Current methods require incubation periods in live animals for weeks of time, extensive laboratory resources and sacrificing a laboratory animal. Culturing Bartonella henselae is not considered an effective, efficient, nor reproducible diagnostic procedure to detect Bartonella infections. While a PCR test of either the organism or its DNA in the test sample at the time of assaying permits identification of Bartonella henselae, PCR assays are only useful during the early infection period. An IFA is not a quantitative assay in that it fails to provide useful data relating to sensitivity and specificity of an infection. IFAs typically rely upon technicians to determine the test results. This fact introduces subjectivity into analyzing and interpreting the test results. IFAs require more time and expensive fluorescent microscopes.

Thus, tools necessary by which to quantify, diagnose, and report reliable results of suspected infections caused by Bartonella henselae are lacking. While enzyme-linked immunosorbent assay (ELISA) offers a quantitative detection assay with greater sensitivity and specificity than IFA, only a limited number of antigens for the Bartonella henselae bacterium have been shown to be useful in the detection of Bartonella. No method is available for quantitatively determining Bartonella henselae infection or of Cat Scratch Disease without the use of live animals.

Accordingly, there is a continuing need for an improved assay to detect Bartonella henselae. A further need exists to minimize the subjectivity of detecting and diagnosing Bartonella henselae. The present disclosure is directed to overcoming these and other deficiencies in the art.

### SUMMARY

In an aspect, provided is method of preparing a calibration sample for detection of anti-Bartonella henselae antibodies in a subject, including obtaining one or more control sample from one or more human control subject infected with Bartonella henselae, isolating antibodies from the one or more control sample to obtain a solution including isolated antibodies, affinity purifying, from the isolated antibodies, antibodies that specifically bind target Bartonella henselae 17 kDa antigen or a fragment thereof to obtain a solution of purified antibodies, and diluting the solution of purified antibodies to a predetermined protein concentration to obtain the calibration sample.

In an example, the target Bartonella henselae 17 kDa antigen includes a sequence of amino acids and the sequence is set out in SEQ ID NO: 1. In another example, the target Bartonella henselae 17 kDa antigen includes a sequence of amino acids and the sequence is set out in SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13. In still another example, the target Bartonella henselae 17 kDa antigen includes a sequence of amino acids and the sequence is set out in SEQ ID NO: 13.

In yet another example, the obtaining one or more control sample from one or more human control subject infected with Bartonella henselae includes obtaining a serum sample from each of a plurality of human control subjects and pooling the serum samples. Another example further includes freezing the calibration sample.

In another aspect, provided is a method of detecting anti-Bartonella henselae antibodies in a test subject, including contacting a peptide with a subject sample, including antibodies, from the test subject, wherein the peptide includes a test Bartonella henselae 17 kDa antigen or a fragment thereof, and detecting a test signal indicating subject sample antibodies binding to the peptide; contacting the peptide with a calibration sample, including antibodies, wherein the calibration sample includes a threshold protein concentration, and detecting a calibration signal indicating calibration sample antibodies binding to the peptide, wherein the calibration sample was prepared by: obtaining one or more control sample from one or more human control subject infected with Bartonella henselae, isolating antibodies from the one or more control sample to obtain a solution including isolated antibodies, affinity purifying, from the isolated antibodies, antibodies that specifically bind a target Bartonella henselae 17 kDa antigen or a fragment thereof to obtain a solution of purified antibodies, diluting the solution of purified antibodies to a predetermined protein concentration to obtain a base calibration sample, and diluting the base calibration sample to obtain the calibration sample having a threshold protein concentration; wherein detecting anti-Bartonella henselae antibodies in the test subject includes detecting a test signal that equals or exceeds the calibration signal.

In an example, the target Bartonella henselae 17 kDa antigen includes a sequence of amino acids and the sequence is set out in SEQ ID NO: 1. In another example, the target Bartonella henselae 17 kDa antigen includes a sequence of amino acids and the sequence is set out in SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13. In still another example, the target Bartonella henselae 17 kDa antigen includes a sequence of amino acids and the sequence is set out in SEQ ID NO: 13.

In yet another example, the obtaining one or more control sample from one or more human control subject infected with Bartonella henselae includes obtaining a serum sample from each of a plurality of human control subjects and pooling the serum samples. In a further example, the threshold protein concentration is about 12.5 ng/ml. In still a further example, the detecting the test signal and the detecting the calibration signal include performing an enzyme-linked immunosorbent assay (ELISA). In yet a further example, the target Bartonella henselae 17 kDa antigen or a fragment thereof includes the test Bartonella henselae 17 kDa antigen or a fragment thereof.

In still another aspect, provided is a kit, including a test Bartonella henselae 17 kDa antigen or a fragment thereof and a calibration sample, wherein the calibration sample includes antibodies that bind a target Bartonella henselae 17 kDa antigen or a fragment thereof; wherein the calibration sample was prepared by: obtaining one or more control sample from one or more human control subject infected with Bartonella henselae, isolating antibodies from the one or more control sample to obtain a solution including isolated antibodies, affinity purifying, from the isolated antibodies, antibodies that specifically bind the target Bartonella henselae 17 kDa antigen or a fragment thereof to obtain a solution of purified antibodies, and diluting the solution of purified antibodies to a predetermined protein concentration to obtain the calibration control sample.

In an example, the target Bartonella henselae 17 kDa antigen includes a sequence of amino acids and the sequence is set out in SEQ ID NO: 1. In still another example, the target Bartonella henselae 17 kDa antigen includes a sequence of amino acids and the sequence is set out in SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13. In yet another example, the target Bartonella henselae 17 kDa antigen includes a sequence of amino acids and the sequence is set out in SEQ ID NO: 13.

In a further example, the obtaining one or more control sample from one or more human control subject infected with Bartonella henselae includes obtaining a serum sample from each of a plurality of human control subjects and pooling the serum samples. In still a further example, the test Bartonella henselae 17 kDa antigen or a fragment thereof is attached to a substrate. In yet a further example, the target Bartonella henselae 17 kDa antigen or a fragment thereof includes the test Bartonella henselae 17 kDa antigen or a fragment thereof.

In yet another aspect, provided is a method of treating a human subject for Bartonella henselae infection, including selecting the subject and administering an antibiotic to the subject, wherein selecting the subject includes obtaining or having obtained a test for detecting anti-Bartonella henselae antibodies in the subject, wherein the test includes: contacting a peptide with a subject sample, including antibodies, from the subject, wherein the peptide includes a test Bartonella henselae 17 kDa antigen or a fragment thereof, and detecting a test signal indicating subject sample antibodies binding to the peptide; contacting the peptide with a calibration sample, including antibodies and a protein concentration of about 12 ng/ml, and detecting a calibration signal indicating calibration sample antibodies binding to the peptide, wherein the calibration sample was prepared by obtaining one or more control sample from one or more human control subject infected with Bartonella henselae, isolating antibodies from the one or more control sample to obtain a solution including isolated antibodies, affinity purifying, from the isolated antibodies, antibodies that specifically bind a target Bartonella henselae 17 kDa antigen or a fragment thereof to obtain a solution of purified antibodies, diluting the solution of purified antibodies to a predetermined protein concentration to obtain a base calibration sample, and diluting the base calibration sample to obtain the calibration sample, and the test signal equals or exceeds the calibration signal.

In an example, the antibiotic includes one or more of a macrolide, a tetracycline, and an aminoglycoside. In another example, the antibiotic is selected from the group consisting of azithromycin, erythromycin, doxycycline, gentamicin, and any combination of two or more of the foregoing. In still another example, the antibiotic is selected from the group consisting of azithromycin, erythromycin and doxycycline, and gentamicin and doxycycline. In yet another example, the target Bartonella henselae 17 kDa antigen includes a sequence of amino acids and the sequence is set out in SEQ ID NO: 1. In a further example, the target Bartonella henselae 17 kDa antigen includes a sequence of amino acids and the sequence is set out in SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13. In another further example, the target Bartonella henselae 17 kDa antigen includes a sequence of amino acids and the sequence is set out in SEQ ID NO: 13.

In still a further example, the obtaining one or more control sample from one or more human control subject infected with Bartonella henselae includes obtaining a serum sample from each of a plurality of human control subjects and pooling the serum samples. In yet a further example, the detecting the test signal and the detesting the calibration control signal include performing an enzyme-linked immunosorbent assay (ELISA). In another example, the target Bartonella henselae 17 kDa antigen or a fragment thereof includes the test Bartonella henselae 17 kDa antigen or a fragment thereof.

In a further aspect, provided is a method of detecting Bartonella henselae infection in a human subject, including contacting a peptide with a subject sample, including antibodies, from the subject, wherein the peptide includes a test Bartonella henselae 17 kDa antigen or a fragment thereof, and detecting a test signal indicating subject sample antibodies binding to the peptide; contacting the peptide with a calibration sample, including antibodies and a protein concentration of about 12 ng/ml, and detecting a calibration signal indicating calibration sample antibodies binding to the peptide, wherein the calibration sample was prepared by obtaining one or more control sample from one or more human control subject infected with Bartonella henselae, isolating antibodies from the one or more control sample to obtain a solution including isolated antibodies, affinity purifying, from the isolated antibodies, antibodies that specifically bind a target Bartonella henselae 17 kDa antigen or a fragment thereof to obtain a solution of purified antibodies, diluting the solution of purified antibodies to a predetermined protein concentration to obtain a base calibration sample, and diluting the base calibration sample to obtain the calibration sample, wherein detecting Bartonella henselae infection includes detecting the test signal equal to or exceeding the calibration signal.

### BRIEF DESCRIPTION OF THE DRAWINGS

These and other features, aspects, and advantages of the present disclosure will become better understood when the following detailed description is read with reference to the accompanying drawings, wherein:

FIG. 1 a non-limiting example of a method by which 17 kDa antigen coated Bartonella henselae plates may be prepared, according to aspects of the present disclosure. 17 kDa antigen may be added to plates and allowed to adsorb overnight. The plates may then be washed, blocked, and dried, preserving antigen for future use.

FIG. 2 depicts an example of an ELISA process.

FIG. 3 depicts 70-sample IFA screened panel when run on an ELISA without a calibration sample, in accordance with aspects of the p[resent disclosure. Only the most positive and negative specimens were able to be analyzed. Most samples were indeterminate due to the lack of calibrator controls.

FIG. 4 depicts an example of serially diluting a calibrator control according to aspects of the present disclosure.

FIG. 5 depicts a non-limiting example of using a calibrator control in a Bartonella henselae ELISA and absorbance values of optical density (OD) readings assigned to samples via spectrophotometry.

FIG. 6 depicts linearity of a non-limiting example of a calibrator control when run with a panel of 70 IFA confirmed sera and QC rules applied to analyze the reliability of the results, in accordance with aspects of the present disclosure.

FIG. 7 depicts a non-limiting example of results including criteria for detecting negative, positive, or equivocal samples based upon the OD value of calibration controls, in accordance with aspects of the present disclosure. In an example, samples with an OD value equal to or greater than that of calibrator 1 are deemed positive for detection. Samples with an OD value equal to or below that of calibrator 2 are deemed negative for detection. Samples with an OD value between calibrators 1 and 2 are deemed equivocal for detection.

### DETAILED DESCRIPTION

This disclosure relates to a method of preparing a calibration sample for detection of anti-Bartonella henselae (B. henselae) antibodies, a method of detecting anti-Bartonella henselae antibodies using a calibration control sample, a kit including a a calibration control sample for detecting anti-Bartonella henselae antibodies, a method for treating a subject for Bartonella henselae infection detected using a calibration control sample, and a method of detecting Bartonella henselae infection.

Provided in the present disclosure is a method to detect B. henselae infection in a quantifiable and non-variable manner. The method also may avoid use of animals to incubate B. henselae for its detection as opposed to conventional methods of detection. The presently disclosed methods, calibration standard, and kit may provide a faster and more economical method to detect 17 kDa antigen and to detect infections with B. henselae compared to conventional methods. Whereas IFA is subjective and prone to erroneous interpretation results, the present disclosure includes calibration standards to accurately assess the degree of a Bartonella henselae infection in a patient. By quantifying a Bartonella henselae infection in a human and obviating animal sacrifice in accordance with aspects of the present disclosure, deficiencies in conventional techniques are overcome, providing an improved tool for Bartonella henselae detection.

A calibration sample, as the term is used herein, includes a sample of anti-17 kDa antibodies affinity purified, from one or more samples from one or more subject, using a 17 kDa antigen or fragment or variant thereof. In an example, the oner or more sample from which the anti-a7 kDa antibodies were purified were human.

B. henselae infection results in generation of antibodies in a subject. An antigen present in B. henselae, referred to as the B. henselae 17 kDa antigen or 17 kDa antigen, has been characterized. U.S. Pat. No. 8,283,439 B2, incorporated herein by reference in its entirety, discloses a 17 kDa antigen and variants thereof and fragments thereof to which antibodies in currently or previously infected subjects bind with high specificity. As disclosed herein, anti-17 kDa antibodies may be affinity purified from subjects presently or previously infected with B. henselae using the 17 kDa antigen or a fragment thereof. Such antibodies may be used as a positive control in a test for whether a person or other subject suspected of having anti-17 kDa antibodies or current or prior infection with B. henselae has such antibodies. For example, anti-17 kDa antibodies affinity purified from one or more human subjects may be included in a calibration sample.

In an example, a calibration sample may be diluted to form serial dilutions, and may be included as one or more comparative controls for a test for detection of antibodies such as an ELISA, or other immunoassay including, without limitation, a radioimmunoassay, a multiplex immunoassay, a counting immunoassay, fluorescence immunoassay, chemiluminescent immunoassay. As disclosed herein, such a calibration control provides a method by which specific immunoglobulin standards may be affinity purified from human samples without sacrificing live animals and provides calibrators and quantitative ranges by which to determine the level of antibodies to Bartonella henselae in a subject. In an example, comparison of an immunoassay result, such as an ELISA result, for detection of anti-17 kDa antigen antibodies in a subject to that of one or more a calibration sample, such as to a calibration curve of results from serial or increasing dilutions of a calibration sample, may distinguish between a true negative result, a false negative result, a false positive result, and a true positive result, as may have been indicated if the immunoassay had been performed without the calibration sample or one or more dilutions thereof.

An immunoassay may include more than one calibration sample, wherein each of the one or more calibration sample has a concentration of affinity-purified anti-17 kDa antigen antibodies that differs from one or more of the other calibration samples included in the immunoassay. As non-limiting examples, an immunoassay may include two or more calibration samples whose concentration of anti-17 kDa antigen antibodies differ from each other, or three or more calibration samples whose concentration of anti-17 kDa antigen antibodies differ from each other, or four or more calibration samples whose concentration of anti-17 kDa antigen antibodies differ from each other, or five or more calibration samples whose concentration of anti-17 kDa antigen antibodies differ from each other, or six or more calibration samples whose concentration of anti-17 kDa antigen antibodies differ from each other, or seven or more calibration samples whose concentration of anti-17 kDa antigen antibodies differ from each other, or eight or more calibration samples whose concentration of anti-17 kDa antigen antibodies differ from each other, or nine or more calibration samples whose concentration of anti-17 kDa antigen antibodies differ from each other, or ten or more calibration samples whose concentration of anti-17 kDa antigen antibodies differ from each other, or elevent or more calibration samples whose concentration of anti-17 kDa antigen antibodies differ from each other, or twelve or more calibration samples whose concentration of anti-17 kDa antigen antibodies differ from each other, or thirteen or more calibration samples whose concentration of anti-17 kDa antigen antibodies differ from each other, or fourteen or more calibration samples whose concentration of anti-17 kDa antigen antibodies differ from each other, or fifteen or more calibration samples whose concentration of anti-17 kDa antigen antibodies differ from each other.

In an example, results from more than one calibration sample used in an immunoassay may be used to construct a calibration curve. For example, a calibration curve may permit comparison of a result from a test sample thereto for determination of a concentration of anti-17 kDa antigen antibody in a test sample, or whether a concentration of anti-17 kDa antigen antibody in a test sample equals, exceeds, or is lower than the concentration of anti-17 kDa antigen antibody in a calibration sample whose result was equals, is lower than, or is higher than, respectively, that for the test sample. In an example, the concentration of anti-17 kDa antigen antibodies in a test sample may be deemed to exceed that for a given calibration sample if the given calibration sample has the highest concentration of anti-17 kDa antigen antibodies of all calibration samples that yielded results that were lower than the result of a test sample. In an example, the concentration of anti-17 kDa antigen antibodies in a test sample may be deemed to be below that for a given calibration sample if the given calibration sample has the lowest concentration of anti-17 kDa antigen antibodies of all calibration samples that yielded results that were higher than the result of a test sample.

For example, an immunoassay test result, such as an ELISA result, may include a signal similar in kind to a signal that conclusively indicates presence of an antibody in a sample but may be of a signal intensity that requires analysis to distinguish between whether the test result is due to non-specific background rather than accurate detection of antibody in the test sample. In accordance with aspects of the present disclosure, a calibration sample may be made and used to provide a definitive threshold of immunoassay result intensity for comparison with an immunoassay test result. Where intensity of a test result at least equals or exceeds a calibration sample intensity threshold, the test result's intensity may confirm that the signal indicates presence of anti-17 kDa antibodies in the sample. Alternatively, where intensity of a test result does is less than a calibration sample intensity threshold, the test result's intensity may fail to confirm that the signal indicates presence of anti-17 kDa antibodies in the sample, or may confirm that the signal indicates absence of anti-17 kDa antibodies in the sample.

In an example, a calibration sample, or one or more dilution or serial or progressive dilutions thereof may permit a quantitative measure of antibodies in a test sample. A calibration sample including predetermined concentrations of protein, for example of antibodies present in a sample of antibodies affinity purified from a sample using a 17 kDa antigen to pull down antibodies, may be used in a method for detection of anti-17 kDa antibodies from one or more sample and tested in parallel with such one or more sample. A protein concentration of a calibration sample, such as a base calibration sample, may be determined by testing the base calibration sample to determine its protein concentration. The base calibration sample or portion thereof may then be diluted to form a calibration sample having a predetermined protein concentration. In an example, a base calibration sample or portion thereof may be serially diluted, or multiple portions thereof may be diluted, to form more than one calibration sample each having a different concentration from other calibration samples. The diluted calibration sample or calibration samples having predetermined protein concentration or concentrations may be tested in an assay, such as an ELISA immunoassay or other immunoassay, to provide results indicating a signal intensity corresponding to the one or more predetermined concentration for the test. For example, a calibration curve may be formed indicating signal intensity corresponding to predetermined protein concentrations and to provide extrapolations of test sample results to conclude protein concentrations below, between, and/or above the predetermined concentrations based on how said test results compared to signal intensities on the calibration curve.

For example, as part of a calibration curve or on its own, a calibration sample may include a predetermined protein concentration, which protein concentration provides a result having a signal intensity that serves as a threshold result in an immunoassay. A test sample may be tested in the assay and the intensity of its result compared to the signal intensity of the threshold calibration sample. If the intensity of the test sample's result equals or exceeds the threshold calibration sample's signal intensity, the test sample's signal intensity indicates presence of anti-17 kDa antibodies in the test sample. In an example, comparing a test sample's signal intensity to one or more calibration sample, in an example including a threshold calibration sample, or in an example including a calibration curve, wherein the one or more calibration sample includes a predetermined protein concentration, indicates a protein concentration in the test sample.

In another aspect the present invention provides for a kit for performing a test, such as an ELISA or other immunoassay, for detection of B. henselae. A kit may include one or more calibration sample including anti-17 kDa antibodies affinity purified from one or more subject, having one or more predetermined protein concentration. A user may use the one or more calibration sample in an immunoassay such as an ELIS or other immunoassay to test a test sample such as a sample taken from a subject suspected of having B. henselae or B. henselae infection or anti-B. henselae antibodies such as anti-17 kDa antibodies. A kit may include a calibration sample and a user may make from it one or more serial or successive dilutions to form a plurality of calibration samples having differing protein concentrations (known, for example, by comparing the amount of dilution to the concentration of protein know in the calibration sample included in the kit). A kit may include a plurality of calibration samples having differing, predetermined protein concentrations. A user may include in an assay to test a test-subject's sample for presence of anti-17 kDa antibodies one or more of the one or more calibration sample, which may include a calibration sample having a threshold level of antibodies.

A kit may also include a 17 kDa antigen, including a fragment of a 17 kDa antigen or variant of a 17 kDa antigen or fragment thereof. A user may use the antigen to prepare a substrate or other reagent for use in an immunoassay for testing a subject's sample for presence of anti-17 kDa antigen antibodies. For example, in an ELISA, a 17 kDa antigen may be attached, adsorbed, affixed, or bound to a plate or other substrate to which a test subject's sample is added, for detection of antibodies in the test subject's sample that may bind to the 17 kDa antigen on the substrate. A kit may include such a 17 kDa antigen or fragment thereof or variant thereof for attachment to a plate or other substrate by a user for use in an immunoassay such as an ELISA (or, for example, another substrate such as a bead for a multiplex immunoassay, etc.). Or a kit may include such a 17 kDa antigen or fragment or variant thereof already attached, adsorbed, affixed, or bound to a plate, beads, or other substrate, for use in an immunoassay.

A kit may also include one or more component or reagent for use in an immunoassay as disclosed herein. A kit may include a positive control calibration sample, having a concentration of anti-17 kDa antigen antibodies above a threshold concentration, a negative control, lacking anti-17 kDa anitgen antibodies or having a concentration of anti-17 kDa antigen antibodies below a lower threshold level or below a level of detection, or optionally including a control peptide, such as bovine serum antigen (BSA) or other control protein, or one or more additional reagents for use in an immunoassay such as an ELISA, including secondary antibodies, substrate for generation of detectable signal in the immunoassay, pH buffers, etc.

For example, a negative control may have a concentration of anti-17 kDa antigen antibodies of about 6.25 ng/ml or lower, or a concentration of anti-17 kDa antigen antibodies of about 6 ng/ml or lower, or a concentration of anti-17 kDa antigen antibodies of about 5.75 ng/ml or lower, or a concentration of anti-17 kDa antigen antibodies of about 5.5 ng/ml or lower, or a concentration of anti-17 kDa antigen antibodies of about 5.25 ng/ml or lower, or a concentration of anti-17 kDa antigen antibodies of about 5 ng/ml or lower, or a concentration of anti-17 kDa antigen antibodies of about 4.75 ng/ml or lower, or a concentration of anti-17 kDa antigen antibodies of about 4.5 ng/ml or lower, or a concentration of anti-17 kDa antigen antibodies of about 4.25 ng/ml or lower, or a concentration of anti-17 kDa antigen antibodies of about 4 ng/ml or lower, or a concentration of anti-17 kDa antigen antibodies of about 3.75 ng/ml or lower, or a concentration of anti-17 kDa antigen antibodies of about 3.5 ng/ml or lower, or a concentration of anti-17 kDa antigen antibodies of about 3.25 ng/ml or lower, or a concentration of anti-17 kDa antigen antibodies of about 3.13 ng/ml or lower, or a concentration of anti-17 kDa antigen antibodies of about 3 ng/ml or lower, or a concentration of anti-17 kDa antigen antibodies of about 2.75 ng/ml or lower, or a concentration of anti-17 kDa antigen antibodies of about 2.5 ng/ml or lower, or a concentration of anti-17 kDa antigen antibodies of about 2.25 ng/ml or lower, or a concentration of anti-17 kDa antigen antibodies of about 2 ng/ml or lower, or a concentration of anti-17 kDa antigen antibodies of about 1.75 ng/ml or lower, or a concentration of anti-17 kDa antigen antibodies of about 1.56 ng/ml or lower, or a concentration of anti-17 kDa antigen antibodies of about 1.5 ng/ml or lower, or a concentration of anti-17 kDa antigen antibodies of about 1.25 ng/ml or lower, or a concentration of anti-17 kDa antigen antibodies of about 1 ng/ml or lower, or a concentration of anti-17 kDa antigen antibodies of about 0.78 ng/ml or lower, or a concentration of anti-17 kDa antigen antibodies of about 0.75 ng/ml or lower, or a concentration of anti-17 kDa antigen antibodies of about 0.5 ng/ml or lower, or a concentration of anti-17 kDa antigen antibodies of about 0.25 ng/ml or lower, or a concentration of anti-17 kDa antigen antibodies of about 0.13 ng/ml or lower, or a concentration of anti-17 kDa antigen antibodies of about 0.07 ng/ml or lower, or a concentration of anti-17 kDa antigen antibodies of about 0.04 ng/ml or lower, or a concentration of anti-17 kDa antigen antibodies of about 0.02 ng/ml or lower, or a concentration of anti-17 kDa antigen antibodies of about 0.01 ng/ml or lower, or a concentration of anti-17 kDa antigen antibodies of about 0 ng/ml.

Such antigen for use in an immunoassay may be referred to herein as a test antigen. A 17 kDa antigen or fragment thereof of variant thereof used for affinity purifying anti-17 kDa antigen antibodies from subjects for preparing a calibration sample, by comparison, may be referred to herein as a target antigen. In an example, a target antigen used for affinity purifying anti-17 kDa antigen antibodies to form a calibration sample may be the same as a test antigen used in an immunoassay including one or more calibration sample for detecting presence of anti-17 kDa antigen antibodies in a test subject. In another example, a target 17 kDa antigen used to affinity purify anti-17 kDa antigen antibodies for a calibration sample may differ from a 17 kDa test antigen for use in an immunoassay with the calibration sample and a test sample for detection of 17 kDa antigen antibodies in the test sample. For example, a target antigen may have an amino acid sequence that includes the amino acid sequence of a test antigen, or a test antigen may include an amino acid sequence that includes the amino acid sequence of a target antigen. Or an amino acid sequence of a test antigen may differ from an amino acid sequence of a target antigen. A target antigen may be one example of a 17 kDa antigen or fragment thereof, or variant thereof, and a test antigen may be a different 17 kDa antigen or fragment thereof, or variant thereof, than that of the target antigen.

In an example, more than one 17 kDa antigen may be used as a target antigen to affinity purify anti-17 kDa antigen antibodies from samples for creating a calibration sample. For example, more than one of a 17 kDa antigen, one or more variant thereof, and one or more fragment thereof, in any combination of two or more of the foregoing, may be used to affinity purify anti-17 kDa antibodies from samples to form a calibration sample. For example, using different, multiple target antigens during affinity purification may increase the scope or overall number of anti-17 kDa antigen antibodies pulled down from a sample.

In an example, more than one 17 kDa antigen may be used as a test antigen to use in an immunoassay with one or more calibration sample for detection of anti-17 kDa antigen antibodies in a test sample. For example, more than one of a 17 kDa antigen, one or more variant thereof, and one or more fragment thereof, in any combination of two or more of the foregoing, may be attached, adsorbed, affixed, or bound to a plate, beads, or other substrate used in such an immunoassay. For example, using different, multiple test antigens in an immunoassay may increase the scope or overall number of anti-17 kDa antigen antibodies detected in a test sample and calibration sample in the immunoassay.

In an example, a kit may include any of the aforementioned calibration samples, made by an affinity purification using one or more than one target 17 kDa antigen. In an example, a kit may include any of the aforementioned test antigens, including one or more 17 kDa antigen, variant thereof, or fragment thereof, and in an example may be attached, adsorbed, affixed, or bound to a plate, beads, or other substrate. And a kit may include any combination of such target antigen and such test antigen.

Non-limiting examples of amino acid sequences of target antigen and test antigen are provided in the following Table 1:

TABLE 1
Amino acid sequences of target 17 kDa antigens and test 17 kDa
antigens

| SEQ ID NO: | Amino acid sequence |
|---|---|
| 1 | MKKYSLVTLLSLFCISHAKAQTATLTDEYYKKALENTQKLDVAKSQT |
| 17 kDa | AESIYESATQTANKIKDINNQLANLKADTKTKPEQLQALQIELTLLQA |
| protein | QLQADTLKIQSLAMIQAKDTKTKEELREEQTQKKHEDLQKQLKEKL |
| | EKSDVQL |
| 2 | KTKPEQLQALQIELTLLQAQLQADTLKIQSLAMIQAKDTKTKEELREE |
| | QTQKKHEDLQKQLKEKLEKSDVQL |
| 3 | KTKPEQLQALQIELTLLQAQLQADTLKIQSLAMIQAKDTKTKEELREE |
| | QTQKKHEDLQKQLKEKLEKSDVRL |
| 4 | KTKPEQLQALQIDLTLLQAQLQADTLKIQSLAMIQAKDTKTKEELRE |
| | EQTQKKHEDLQKQLKEKLEKSDVQL |
| 5 | KTKPEQLQALQIELTVLQAQLQADTLKIQSLAMIQAKDTKTKEELRE |
| | EQTQKKHEDLQKQLKEKLEKSDVQL |
| 6 | KTKPEQLQALIELTLLQAQLQADTLKIQSLAMIQAKDTKTKEELREEQ |
| | TQKKHEDLQKQLKEKLEKSDVQL |
| 7 | KTKPEQLQALQIELTLLVQAQLQADTLKIQSLAMIQAKDTKTKEELR |
| | EEQTQKKHEDLQKQLKEKLEKSDVQL |
| 8 | EDLQKQLKEKLEKSDVRL |
| 9 | DDLQKQLKEKLEKSDVRL |
| 10 | EDIQKQLKEKLEKSDVRL |
| 11 | EDLQKQVKEKLEKSDVRL |
| 12 | LKQDEQKLEKLEKSDVRL |
| 13 | TKPEQLQALQIELTLLQAQLQADTLKIQSLAMIQAKDTKTKEELREEQ |
| | TQKKHEDLQKQLKEKLEKSDVQL |

Amino acid sequences of target antigen and test antigen are disclosed herein and also in U.S. Pat. No. 8,283,439 B2, incorporated herein by reference in its entirety. Human subjects with B. henselae or Cat Scratch disease or B. henselae infection, presently or historically, may produce antibodies that specifically bind to a 17 kDa antigen having an amino acid sequence such as one or more of those set out in Table 1.

In an example, a target or test 17 kDa antigen or variant thereof or fragment thereof may include one or more amino acid or sequence thereof in addition to the one or more amino acid or sequence thereof that corresponds to an amino acid sequence as may be produced by B. henselae. For example, a target 17 kDa antigen as in accorance with aspects of the present disclosure, or a test 17 kDa antigen in accordance with aspects of the present disclosure, may include, in addition to a sequence of amino acids that corresponds to a sequence of amino acids of a 17 kDa antigen produced by B. henselae during infection, may include one of more additional amino acid sequence, C-terminal thereto, N-terminal thereto, or both. As a non-limiting example, a 17 kDa test antigen or 17 kDa target antigen may include various sequences of tags for identification or separation of a 17 kDa test antigen or 17 kDa target antigen. Some non-limiting examples of such peptide tags, known to skilled persons in the relevant fied, include and ALFA-tag, an AviTag, a C-tag, a Calmodulin-tag, an ICAPTAG™, a polyglutamate tag, a polyarginine tag, an E-tag, a FLAG-tag, an HA-tag, a HIS-tag, a Myc-tag, an NE-tag, a Rh01D4-tag, an S-tag, an SBP-tag, a Softag, a Spot-tag, a Strep-tag, a T7-tag, a TC tag, a Ty tag, a V5 tag, a VSV-tag, an Xpress tag, or other one or more tag or other one or more amino acid sequence in addition to an amino acid sequence corresponding to an amino acid sequence of a 17 kDa protein expressed by B. henselae.

One or more amino acid in the sequences in Table 1 for a target 17 kDa antigen or a test 17 kDa antigen may be substituted with another amino acid. For example, an amino acid of one type of class may be substituted by another amino acid in the same class, or having similar chemical or physical properties, as would be understood by skilled persons, in what is referred to as a conservative substitution. A conservative substitution is one in which an amino acid is substituted for another amino acid that has similar properties, such that one skilled in the art of peptide chemistry would expect the secondary structure and hydropathic nature of the polypeptide to be substantially unchanged. In general, a substitution of one amino acid within the following groups for another amino acid within the following groups represents a conservative substitution: (1) ala, pro, gly, glu, asp, gln, asn, ser, thr; (2) cys, ser, try, thr; (3) val, ile, leu, met, ala, phe; (4) lys, arg, his; and (5) phe, tyr, trp, his.

A target 17 kDa antigen or test 17 kDa antigen may include an amino acid sequence as set out in Table 1, or may include one or more substitution, including a conservative amino acid substitution. Thus, a target 17 kDa antigen or a test 17 kDa antigen may include an amino acid sequence that is 100% homologous with an amino acid sequence as set out in Table 1, meaning it includes an amino acid sequence as set out in Table 1. Or it may include an amino acid sequence that has less than 100% homology with an amino acid sequence as set out in Table 1. For example, it may include one amino acid substitution, such as a conservative amino acid substitution, compared to a sequence as set out in Table 1. For example, it may have 1, 2, 3, 4, 5, 6, 7, 8, 9, 10, 11, 12, 13, 14, 15, 16, 7, 18, 19, 20, or more amino acid substitutions, including 1, 2, 3, 4, 5, 6, 7, 8, 9, 10, 11, 12, 13, 14, 15, 16, 7, 18, 19, 20, or more conservative amino acid substitutions, provided it still binds anti-17 kDa antigen antibodies produced by a subject such as a human subject who has B. henselae or previously had B. henselae or has Cat Scratch disease or previously had Cat Scratch disease or who has anti-17 kDa antigen antibodies.

One or more amino acid in a test 17 kDa antigen or target 17 kDa antigen may be an R-amino acid or an L-amino acid. One or more amino acid in a test 17 kDa antigen or target 17 kDa antigen may be a standard amino acid (i.e., selected from Alanine, Arginine, Asparagine, Aspartic Acid, Cysteine, Glutamic acid, Glutamine, Glycine, Histidine, Isoleucine, Leucine, Lysine, Methionine, Phenylalanine, Proline, Serine, Threonine, Tryptophan, Tyrosine, Valine, Selenocysteine, N-formylmethionine, and Pyrrolysine).

The term “amino acid” or “any amino acid” as used here refers to any and all amino acids, including naturally occurring amino acids (e.g., α-amino acids), unnatural amino acids, modified amino acids, and non-natural amino acids. It includes both D- and L-amino acids. Natural amino acids include those found in nature, such as, e.g., 23 aforementioned amino acids that combine into peptide chains to form the building-blocks of a vast array of proteins. These are primarily L stereoisomers, although a few D-amino acids occur in bacterial envelopes and some antibiotics. “Unnatural” or “non-natural” amino acids are non-proteinogenic amino acids (i.e., those not naturally encoded or found in the genetic code) that either occur naturally or are chemically synthesized. Over 140 unnatural amino acids are known and thousands of more combinations are possible. Examples of “unnatural” amino acids include R-amino acids (03 and 02), homo-amino acids, proline and pyruvic acid derivatives, 3-substituted alanine derivatives, glycine derivatives, ring-substituted phenylalanine and tyrosine derivatives, linear core amino acids, diamino acids, D-amino acids, alpha-methyl amino acids and N-methyl amino acids. Unnatural or non-natural amino acids also include modified amino acids. “Modified” amino acids include amino acids (e.g., natural amino acids) that have been chemically modified to include a group, groups, or chemical moiety not naturally present on the amino acid.

Examples of such amino acids that may be substituted for 1, 2, 3, 4, 5, 6, 7, 8, 9, 10, 11, 12, 13, 14, 15, 16, 7, 18, 19, 20, or more amino acid in a test or target 17 kDa antigen as set out in Table 1 include (1-Methyl)Histidine, D-2-Naphthylalanine, (D)-alpha-Me-Phenylalanine, (D)-alpha-Me-Tyrosine, NMe(D)Tyr or N-Me-(D)Tyrosine, D-Ornithine, (D)-(3-Amino)phenylalanine, (D)-(4-Amino)phenylalanine, N-Pyrazol-3-yl-Asparagine, N-Pyrazol-4-yl-Asparagine, N-indol-5-yl-Asparagine, N-(imidazo-2-yl)methyl-Asparagine, N—CH₂CH₂CONH₂-Asparagine, N-Ph(2-NH₂)-Asparagine, N-Ph(3-NH₂)-Asparagine, N-Ph(4-NH₂)-Asparagine, N-benzyl-Asparagine, N-Ph-Asparagine, N-piperidin-4-yl-Asparagine, N-Pyrrolidin-3-yl-Asparagine, L-1,2,3,4-tetrahydro-norharman, 1-Aminoindane-1-carboxylic acid, L-1-Naphthylalanine, 2,5,7-Tris-tert-butyl-L-tryptophan, 2-Aminoindane-2-carboxylic acid, L-2-Naphthylalanine, L-2-Pyridylalanine, L-3-Pyridylalanine,

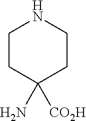

(4-amino-4-carboxy-piperidine),

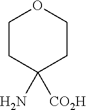

(4-amino-4-carboxy-tetrahydropyran), L-4-Pyridylalanine, 4-L-Pyridylalanine,

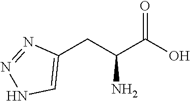 [SMILES: N[C@@H](Cc1c[nH]nn1)C(=O)O]

(4-triazole-alanine), 5-Hydroxy-L-Tryptophan,

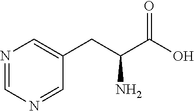 [SMILES: N[C@@H](Cc1cncnc1)C(=O)O]

(5-pyrimidine-alanine), 6-Chloro-L-Tryptophan, 2-Aminobutyric acid,

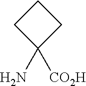

(1-aminocyclobutanecarboxylic acid),

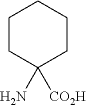

(1-aminocyclohexanecarboxylic acid),

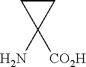

(1-aminocyclopropylcarboxylic acid),

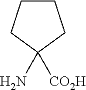

(1-aminocyclopentanecarboxylic acid), (2-aminoethoxy)acetic acid, 3-(2-aminoethoxy)propanoic acid, 6-aminohexanoic acid, 2-aminoisobutyric acid, α-Methyl-L-Asparagine, α-Methyl-L-Glutamine, alpha-methyl Glutamic Acid, α-Methyl-(4-Fluoro)phenylalanine, alpha-methyl-L-Proline, L-azetidine-2-carboxylic acid, L-4,4′-Biphenylalanine, L-Cavanine, Cyclohexyl-L-alanine, L-Citrulline, Carboxamide,

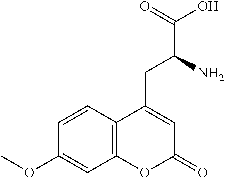 [SMILES: COc1ccc2c(C[C@H](N)C(=O)O)cc(=O)oc2c1]

(Cyclopentyl-L-alanine), L-cyclobutylalanine, (2- or beta-)-cyclohexyl-L-Alanine, L-Diaminobutyric acid, N-Acetyl-L-diaminobutyric acid, L-Diaminopropionic acid, N-Acetyl-L-Diaminopropionic acid,

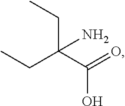

### 2,6-DimethylTyrosine, Dithiothreitol,

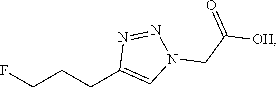 [SMILES: O=C(O)Cn1cc(CCCF)nn1]

Gamma-Carboxy-L-Glutamic acid, N-allyl-L-Glycine, N-Cyclohexylmethyl-L-Glycine, N-Isobutyl-L-Glycine, L-homoArginine, L-homocyclohexylalanine,

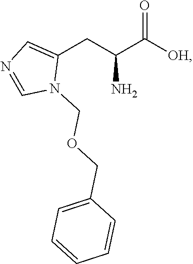 [SMILES: N[C@@H](Cc1cncn1COCc1ccccc1)C(=O)O]

### (3-Methyl)Histidine,

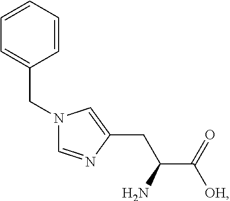 [SMILES: N[C@@H](Cc1cn(Cc2ccccc2)cn1)C(=O)O]

L-homoLeucine, homo-L-Lysine, Homophenylalanine, 3,4-dimethoxy-L-homophenylalanine, L-homoSerine, 4-Hydroxy-L-Proline, Iso-Propyl, N-ε-acetyl-L-Lysine,

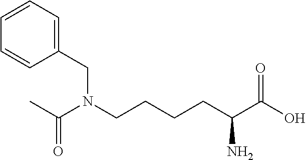 [SMILES: CC(=O)N(CCCC[C@H](N)C(=O)O)Cc1ccccc1]

(N^{ε}-acetyl-N^{ε}-benzyl-L-Lysine or Lys(N-acetyl-N-benzyl)),

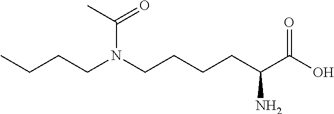 [SMILES: CCCCN(CCCC[C@H](N)C(=O)O)C(C)=O]

(N^{ε}-acetyl-N^{ε}-butyl-L-Lysine or Lys(N-acetyl-N-butyl)), N—(C(O)2-Allyl)-Lysine, N-c-trifluoroacetyl-L-Lysine, N-Trifluoroacetyl-Lysine, Lys(CO-cyclopropyl), N—(C(O)-Et)-Lysine, N-c-[C(O)-i-Bu]-L-Lysine, N—(C(O)-i-Pr)-Lysine, Lys(CO-pentyl), N—(C(O)-n-Pr)-Lysine, N-c-[C(O)-t-Bu]-L-Lysine, N—(C(O)-t-Bu)-Lysine,

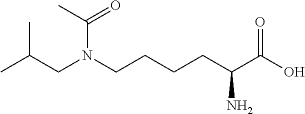 [SMILES: CC(=O)N(CCCC[C@H](N)C(=O)O)CC(C)C]

(N^{ε}-acetyl-N^{ε}-isobutyl-L-Lysine or Lys(N-acetyl-N-isobutyl)),

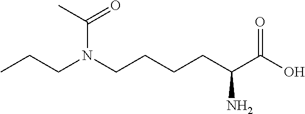 [SMILES: CCCN(CCCC[C@H](N)C(=O)O)C(C)=O]

(N^{ε}-acetyl-N^{ε}-propyl-L-Lysine or Lys(N-acetyl-N-propyl)), N-ε-[R]-L-Lysine (exemplary R=Aib, bAla, IVA, Ala, cyclohexanoic, octanoic, —C(O)CH₂Ph, trifluorpropionic, Gly, acetyl, trifluoroacetyl, etc.), N-2-aminoanilinyl-L-asparagine (L-asparagine, N-2-aminoanilinyl), N-3-aminoanilinyl-L-asparagine (L-asparagine, N-3-aminoanilinyl), N-4-aminoanilinyl-L-asparagine (L-asparagine, N-4-aminoanilinyl), N-Alkyl-L-asparagine (L-asparagine, N-alkyl), (L) H₂N—C(H)(CO₂H)—CH₂—C(O)—NH(Alkyl), N-4-aminobutyl-L-asparagine (L-asparagine, N-4-aminobutyl), N-anilinyl-L-asparagine (L-asparagine, N-anilinyl), N-butyl-L-asparagine (L-asparagine, N-butyl), N-benzyl-L-asparagine (L-asparagine, N-benzyl), N-cyclohexyl-L-asparagine (L-asparagine, N-cyclohexyl), N-cyclopropyl-L-asparagine (L-asparagine, N-cyclopropyl), N-ethyl-L-asparagine (L-asparagine, N-ethyl), N-isobutyl-L-asparagine (L-asparagine, N-isobutyl), N-isopropyl-L-asparagine (L-asparagine, N-isopropyl), N-methyl-L-asparagine (L-asparagine, N-methyl), N-piperidinyl-L-asparagine (L-asparagine, N-piperidinyl), N-tert-butyl-L-asparagine (L-asparagine, N-tert-butyl), N₃—CH₂—COOH, L-Norleucine, N-Methyl-L-Alanine, N-Methyl-L-Arginine, N-Methyl-L-Asparagine, N-Methyl-L-Glutamine, N-Methyl-Lysine, N-Methyl-L-Lysine, N-E-Acetyl-N-Methyl-L-lysine, N-Methyl-L-Tryptophan, N-Methyl-beta-Alanine, L-Octylglycine, L-Ornithine,

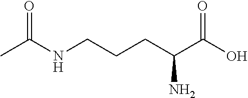

(N-Acetyl-L-ornithine), L-Penicillamine, L-Penicillamine(sulfoxide), 4-(3,4-dimethoxy)phenylalanine, 2,4-dimethyl-L-phenylalanine, 3,4-dichloro-L-phenylalanine, 3,4-dimethoxy-L-phenylalanine, 3,5-difluoro-L-phenylalanine, 4-(2-(N-t-Boc)-aminoethoxy)phenylalanine, 4-bromo-L-phenylalanine, 4-Trifluoromethyl-L-Phenylalanine, 4-cyano-L-phenylalanine, 4-Carboxy-L-phenylalanine, 4-Carbamoyl-L-phenylalanine, 4-Fluoro-L-Phenylalanine, 4-Guanidine-L-Phenylalanine, 4-methyl-L-phenylalanine, 4-azidophenylalanine, 4-amino-L-phenylalanine, O-Allyl-L-Tyrosine, 0-Benzyl-L-tyrosine, 4-Methoxy-L-phenylalanine, 4-Phenoxy-L-phenylalanine, pentafluoro-L-phenylalanine, t-butyl-L-phenylalanine, a-Methyl-4-(2-aminoethoxy)phenylalanine,

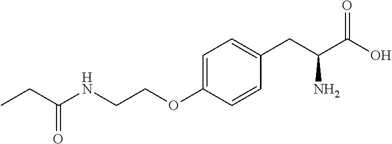 [SMILES: CCC(=O)NCCOc1ccc(C[C@H](N)C(=O)O)cc1]

L-4-[(Ac—NH—CH₂CH₂—O)]-Ph-CH₂—C(H)(NH₂)CO₂H or 4-(2-acetylaminoethoxy)-L-phenylalanine,

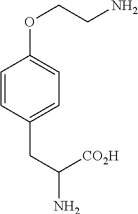 [SMILES: NCCOc1ccc(CC(N)C(=O)O)cc1]

(4-(2-aminoethoxy)-L-phenylalanine), (4-aminomethyl)Phenylalanine,

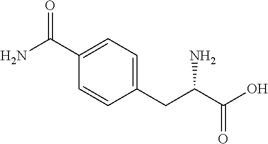 [SMILES: NC(=O)c1ccc(C[C@H](N)C(=O)O)cc1]

(Phe[4-(2-(N-(4-hydroxy-3-methylphenyl)propionylamino)ethoxy)), Phe[4-(2-(N-propionylamino)ethoxy)]-, Phe[4-(2-aceylaminoethoxy)]-, 4,4-difluoro-L-Proline, (S)-2-amino-3-(quinolin-2-yl)propanoic acid or 2-quinolinylalanine,

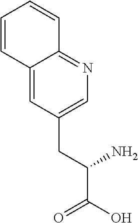 [SMILES: N[C@@H](Cc1cnc2ccccc2c1)C(=O)O]

((S)-2-amino-3-(quinolin-3-yl)propanoic acid or 3-quinolinylalanine), Sarcosine or N-methylglycine,

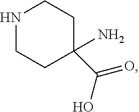

3-(tert-butyl)-L-Alanine-OH, tert-butyl-glycine, 4Aza4-aza-tryptophan, 7Aza7-aza-tryptophan, 3-t-butyl-L-tyrosine or L-(4-difluoromethoxy)Phenylalanine, 4-fluoro-L-tryptophan, (5-Carboxamido)-L-Tryptophan, 5-cyano-L-tryptophan, 5-Phenyl-Tryptophan, 6-Phenyl-Tryptophan, 7-(naphth-1-yl)-Tryptophan, 7-(2-Fluoro-phenyl)Tryptophan, 7-(naphth-2-yl)-Tryptophan, 7-(3,5-di-tert-butylphenyl)-Tryptophan, 7-(biphenyl-3-yl)-Tryptophan, 7-(3-carboxamidophenyl)-Tryptophan, 7-(3-trifluoromethylphenyl)-Tryptophan, 7-(3-isopropylphenyl)-Tryptophan, 7-(3-methylphenyl)-Tryptophan, 7-(3-trifluoromethoxyphenyl)-Tryptophan, 7-(3-MethoxyPhenyl)-Tryptophan, 7-(3-pyrazol-1-yl)-Tryptophan, 7-(4-Anthracen-5-yl)Tryptophan, 7-(biphenyl-4-yl)-Tryptophan, 7-(4-carboxamidophenyl)-Tryptophan, 7-(quinoline-4-yl)-Tryptophan, 7-(Phenanthren-5-yl)Tryptophan, 7-cyano-L-tryptophan, 7-(imidazopyridinyl)-Tryptophan, 7-(indazol-5-yl)-Tryptophan, 7-Phenyl-Tryptophan, 7-(pyrimidin-5-yl)-Tryptophan, 7-thienyl-Tryptophan, L-β-Alanine, L-β-Glutamic acid, L-β-homoglutamine, L-β-homoglutamic acid, L-β-homophenylalanine, L-β-homoproline, L-β-homoTryptophan, alpha-methyl-L-Arginine, alpha-methyl-L-Cysteine, alpha-methyl-L-Leucine, ε-acetyl-alpha-methyl-L-Lysine, alpha-methyl-L-Lysine, alpha-methyl-L-Ornithine, alpha-methyl-L-Phenylalanine, alpha-methyl-L-Tryptophan, alpha-methyl-L-Tyrosine, α-DiethylGlycine, beta-Alanine, beta homo-L-Alanine, beta homo-L-Leucine, beta homo-L-Trptophan, beta homo-L-Tyrosine, and beta homo-L-Valine, provided a test or target 17 kDa antigen binds anti-17 kDa antigen antibodies produced by a subject such as a human subject who has B. henselae or previously had B. henselae or has Cat Scratch disease or previously had Cat Scratch disease or who has anti-17 kDa antigen antibodies.

A sample from a subject from which anti-17 kDa antigen antibodies may be affinity purified for formation od a calibration curve as described herein may be any type of sample that ay include antibodies, including blood, serum, saliva, sweat, urine feces, or any other sample of tissue of bodily fluids. In an example, a sample may be serum or plasma. One or more sample may be taken from a subject for affinity purification therefrom of anti-17 kDa antigen antibodies, and may be pooled before antibodies are isolated therefrom. In an example, antibodies are first isolated from samples and samples are then pooled before affinity purification therefrom of anti-17 kDa antigen antibodies. In another example, anti-17 kDa antigen antibodies are affinity purified from antibodies isolated from each of multiple samples, and affinity purified antibodies from each sample then pooled.

In another example, one or more sample may be taken from each of more than one subject for affinity purification therefrom of anti-17 kDa antigen antibodies, and may be pooled before antibodies are isolated therefrom. In an example, antibodies are first isolated from samples and samples are then pooled before affinity purification therefrom of anti-17 kDa antigen antibodies. In another example, anti-17 kDa antigen antibodies are affinity purified from antibodies isolated from each of multiple samples, and affinity purified antibodies from each sample then pooled.

A subject from whom a sample is obtained for affinity purification of anti-17 kDa antigen antibodies therefrom may be any subject such as a human subject who is confirmed as having B. henselae or previously having had B. henselae or having Cat Scratch disease or previously having had Cat Scratch disease, or otherwise and in any event having anti-17 kDa antigen antibodies. In an example, a subject or all subjects from whom samples are obtained for affinity purification therefore of anti-17 kDa antigen antibodies are human subjects. In an example, a serum sample is obtained from each of a plurality of human subjects with B. henselae, antibodies are isolated therefrom to form a solution of isolated antibodies, and anti-17 kDa antibodies are affinity purified therefrom for each subject's sample to form solutions of purified antibodies, which are pooled. The protein concentration of the pooled anti-a7 kDa antibodies may be ascertained. If the concentration is higher than a desired protein concentration for a base calibration sample, the pooled sample may be diluted to a preferred, predetermined protein concentration to form a base calibration sample. A pooled sample, or individual subject's purified samples, may be frozen before a protein concentration is determined, or after a protein concentration is determined, or after dilution to form a calibration sample having a predetermined protein concentration.

A predetermined protein concentration to which a calibration sample is diluted to form a base calibration sample may be any desired concentration. A predetermined concentration may be from about 10 ng/ml to about 20 mg/ml. A predetermined concentration may be about 10 ng/ml, or about 20 ng/ml, or about 30 ng/ml, or about 40 ng/ml, or about 50 ng/ml, or about 60 ng/ml, or about 70 ng/ml, or about 80 ng/ml, or about 90 ng/ml, or about 100 ng/ml, or about 200 ng/ml, or about 300 ng/ml, or about 400 ng/ml, or about 500 ng/ml, or about 600 ng/ml, or about 700 ng/ml, or about 800 ng/ml, or about 900 ng/ml, or about 1,000 ng/ml, or about 1,500 ng/ml, or about 2,000 ng/ml, or about 2,500 ng/ml, or about, 3,000 ng/ml, or about 3,500 ng/ml, or about 4,000 ng/ml, or about 4,500 ng/ml, or about 5,000 ng/ml, or about 5,500 ng/ml, or about 6,000 ng/ml, or about 6,500 ng/ml, or about 7,500 ng/ml, or about 8,000 ng/ml, or about 8,500 ng/ml, or about 9,000 ng/ml, or about 9,500 ng/ml, or about 10,000 ng/ml, or about 11,000 ng/ml, or about 12,000 ng/ml, or about 13,000 ng/ml, or about 14,000 ng/ml, or about 15,000 ng/ml, or about 16,000 ng/ml, or about 17,000 ng/ml, or about 18,000 ng/ml, or about 19,000 ng/ml, or about 20,000 ng/ml, or higher, or any amount between any of the foregoing amounts.

One or more calibration sample may be diluted from the base calibration sample or portion thereof, to form a calibration sample having a threshold protein concentration, which may include forming multiple calibration samples having differing predetermined protein concentrations, such as for creating a calibration curve when used in an assay to test for presence of anti-17 kDa antigen antibodies. Calibration sample, diluted from a base calibration sample, may have a predetermined protein concentration of from about 0.1 ng/ml to about 100 ng/ml. A calibration sample diluted from a base calibration sample may have a predetermined protein concentration of about 0.1 ng/ml, or about 0.2 ng/ml, or about 0.3 ng/ml, or about 0.4 ng/ml, or about 0.5 ng/ml, or about 0.6 ng/ml, or about, 0.7 ng/ml, or about, 0.8 ng/ml, or about 0.9 ng/ml, or about 1 ng/ml, or about 2 ng/ml, or about 3 ng/ml, or about 4 ng/ml, or about 5 ng/ml, or about 6 ng/ml, or about 7 ng/ml, or about 8 ng/ml, or about 9 ng/ml, or about 10 ng/ml, or about 11 ng/ml, or about 12 ng/ml, or about 13 ng/ml, or about 14 ng/ml, or about 15 ng/ml, or about 16 ng/ml, or about 17 ng/ml, or about 18 ng/ml, or about 19 ng/ml, or about 20 ng/ml, or about 21 ng/ml, or about 22 ng/ml, or about 23 ng/ml, or about 24 ng/ml, or about 25 ng/ml, or about 26 ng/ml, or about 27 ng/ml, or about 28 ng/ml, or about 29 ng/ml, or about 30 ng/ml, or about 35 ng/ml, or about 40 ng/ml, or about 45 ng/ml, or about 50 ng/ml, or about 55 ng/ml, or about 60 ng/ml, or about 70 ng/ml, or about 75 ng/ml, or about 80 ng/ml, or about 85 ng/ml, or about 90 ng/ml, or about 95 ng/ml, or about 100 ng/ml, or higher, or any amount between any of the foregoing amounts. A calibration sample diluted from a base calibration sample may have a predetermined protein concentration of about 0.78 ng/ml, or about 1.56 ng/ml, or about 3.13 ng/ml, or about 6.25 ng/ml, or about 12.5 ng/ml, or about 25 ng/ml. In an example, a threshold protein concentration is about 12.5 ng/ml. In an example, one or more calibration sample having differing predetermined protein concentrations from each other may be rank ordered and numbered, such as 1 through 2, or 3, or 4, or 5, or 6, or 7, or 8, or 9, or 10, or any number of calibration samples, each representing a dilution of a base calibration sample. One of which may include a threshold protein concentration.

A calibration sample or calibration samples as disclosed herein may be used in an immunoassay for detection of presence of anti-17 kDa antigen antibodies in a subject. In an example, an assay may result in a detectable signal (e.g., optical density (OD), radiological, fluorescent, chemiluminescent, etc.) in a subject's test sample and in one or more calibration sample. The signal may have an intensity positively correlated with concentration of protein in a calibration sample. The assay may also include a non-specific contribution to signal intensity, such as due to non-specific background or other error. A predetermined protein concentration of a calibration sample whose signal intensity would signify, if detected as a result for a subject's test sample, a high probability of a true positive indication of presence of anti-17 kDa antigen antibodies in said test sample, may be set as a threshold protein concentration. When an immunoassay is performed including one or more calibration sample, including a calibration sample having a threshold protein concentration, and a subject's test sample, if the signal intensity of the result from the subject's test sample equals or exceeds the signal intensity detected for the calibration sample having the threshold protein concentration, the result may confirmatively signify the presence and detection anti-17 kDa antigen antibodies in the subject's test sample, irrespective of possible background or other potentially “false positive” contributions of the assay. In another example, a calibration sample may have a negative threshold protein concentration, wherein detection of a signal intensity of such a sample in such an assay or a lower intensity confirmatively signifies an absence of anti-17 kDa antigen antibodies in the subject's test sample.

In an example, a subject's test sample result equaling or exceeding that of a calibration sample having a protein threshold may be determined to be infected with B. henselae.

In an example, a subject may be suspected of having B. henselae or Cat Scratch disease and confirmation may be desired before a treatment therefor is administered. Examples of treatments of B. henselae infection or Cat Scratch disease include antibiotics. Examples include one or more of a macrolide, a tetracycline, an aminoglycoside, and any combination of two or more of the foregoing. Examples include one or more of azithromycin, erythromycin, doxycycline, gentamicin, and any combination of two or more of the foregoing.

A macrolide may include azithromycin, clarithromycin, fidaxomicin, or erythromycin. A tetracycline may include demeclocycline, doxycycline, eravacycline, minocycline, omadacycline, oxytetracycline, sarecycline, or tetracycline An aminoglycoside may include paromomycin, amikacin, plazomicin, tobramycin, neomycin, kanamycin, gentamicin, or amikacin.

An example includes any one or two or more antibiotics, including of any one or two or more of the foregoing antibiotics given in combination, including as a non-limiting example gentamicin and doxycycline given in combination.

In an example, a physician or other health care professional may order or obtain a test for anti-17 kDa antigen antibodies in a subject having or suspected of having B. henselae or Cat Scratch disease before determining whether or not to administer treatment for B. henselae infection or Cat Scratch disease. In an example a subject may have a test for anti-17 kDa antigen antibodies performed in accordance with aspects of the present disclosure and provide them to a physician or other health care provider or office, and the physician or other health care provider or office may, on the basis of a result, treat the subject such as with an antibiotic as disclosed herein upon the conclusion, based on results of the test, that the subject does or may have B. henselae infection or Cat Scratch disease or anti-17 kDa antigen antibodies.

Anti-17 kDa antibodies as described herein may be of any type of immunoglobulin, such as IgG IgM. Commercial kits are available for isolating antibodies fro ma subject's sample, such as isolating IgG antibodies from a subject's serum or isolating IgM antibodies from a subject's sample. Such methods known to skilled persons may be used in accordance with aspects of the present disclosure to prepare a solution of isolated antibodies from a subject's sample, from which anti-17 kDa antigen antibodies may be affinity purified. Furthermore, commercial kits, products, reagents, and methods are available for using an antigen to affinity purify antibodies that bind the antigen from a sample, such as from a solution of isolated antibodies, to form a solution of purified antibodies, i.e. affinity purified antibodies pulled down using an anti-17 kDa antigen as disclosed herein. A skilled person may use such a method, non-limiting examples of which are disclosed herein, to prepare a calibration sample using a target 17 kDa antigen in accordance with aspects of the present discourse. A non-limiting example includes an affinity gel column, a column of resin or cross-linked agarose gel used in the process of affinity purification. Commercial kits, products, reagents, and methods are available for attaching an antigen to a substrate such as a plate, beads, or other substrate, for using the antigen in an immunoassay such as an ELISA, and may be used by a user of a kit disclosed herein wherein the kit includes a test 17 kDa antigen and uses it in an immunoassay including binding the antigen to a substrate such as a plate or wells or beads or other substrate.

As used herein the term “ELISA” refers, in a non-limiting example, to an enzyme-linked immunosorbent assay wherein an antigen or antibody is adsorbed onto the surface of a solid phase such as a and a sample is added. A tagged detection antibody is added to said plate followed by a substrate (TMB). If the target antigen or antibody is present, the addition of the substrate will catalyze a colorimetric reaction giving an optical density signal whose intensity may be directly proportional to the level of anti-antigen antibodies in the sample.

When used herein, the term about, followed by a number, means the amount indicated may be within a range of from 10% below the number to 10% above the number. For example, as used herein, the term “about 10” would mean from 9 to 11.

### EXAMPLES

The following examples are intended to illustrate particular embodiments of the present disclosure, but are by no means intended to limit the scope thereof.

### Example 1: Expressing and Purifying 17 kDa Antigen

17 kD antigen fragment whose amino acid sequence is set forth in SEQ ID NO. 14 was obtained after culturing Escherichia coli cells containing the pET-30Ek/LIC vector (EMD Millipore) encoding 17-kDa antigen fragment. This expression vector drives expression of a peptide of interest plus an amino acid sequence N-terminal thereto. SEQ ID NO: 13 sets out the amino acid sequence of a 17 kDa target antigen and a 17 kDa test antigen as used in the examples herein, which also included, N-terminal to the sequence in SEQ ID NO: 13, additional amino acids as encoded within the open reading frame of the pET-30Ek/LIC vector.

The cells were grown at 37° C., 220 rpm in 2 L Luria-Bertani medium containing Kanamycin at a concentration of 30 μg/mL (LB-Kan-30). Expression of the protein was induced once the cells had reached the exponential growth phase by the addition of isopropylthio-β-galactoside (IPTG) at a concentration of 1 mM/L, thus triggering the expression of the lac operon.

The cells were lysed via chemical means using 5 mL Novagen® BugBuster® Protein Extraction Reagent per gram of wet cell paste, supplemented with protease inhibitors. The resulting inclusion body cell pellets were washed with 6 volumes of 1:10 diluted BugBuster® Protein Extraction Reagent in ddH₂O.

The 17 kDa antigen was extracted by denaturing the inclusion bodies with a bind buffer containing 6M Urea. The fragment was purified via nickel-His tag chromatography utilizing 4 mL Millipore Sigma Ni-NTA His-bind Resin. The product of this purification was assayed via SDS-PAGE utilizing a Bio-Rad® 4-12% Criterion™ XT Bis-Tris protein gel and the Bio-Rad® XT MES Buffer Kit.

The most positive fractions, 4-8, were pooled and dialyzed overnight against 4 L of 1×PBS/0.02% glycerol solution in a Thermo-Scientific™ 7K MWCO Slide-a-Lyzer™ cassette. The concentration of said product was ascertained by running a 1:10 dilution of the dialyzed product on a ThermoFisher Pierce™ bicinchoninic acid (BCA) assay.

The purity of the quantified product was verified using Image-J image scanning software (NIH). In total, 4.8 mL of the 17 kDa antigen was obtained at a concentration of 4.44 mg/mL. The final yield was 21.29 mg at 86.73% purity.

### Example 2: Using the 17 kDa Antigen to Prepare the Coated Plates

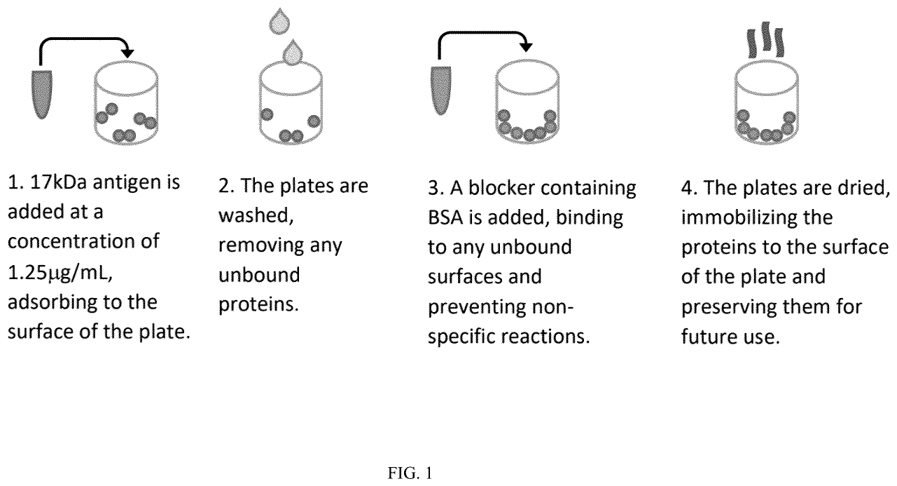

Purified test 17 kDa antigen was diluted to a concentration of 1.25 μg/mL in 1×PBS (phosphate-buffered saline) and coated on the surface of the wells of a solid-phase support. The support used for the purpose of these experiments was a Nunc™ 96-well maxisorp polystyrene microtiter plate (Thermo-Fisher Scientific™). The microtiter plates were covered with a seal and incubated at 4° C.+2° C. overnight, allowing the antigens to adsorb to the surface of the plate. The wells of the plate were washed with 350% L 1×PBS/0.05% Tween twice, no residence time. The plates were tapped onto a paper towel to absorb any excess liquid prior to adding 200 μL blocking solution containing bovine serum albumin (BSA) to each well of the microtiter plate. The plates were covered and incubated at room-temperature (20° C.±5° C.) for four hours. After incubation, the blocker was dumped out and the plates were dried in a vacuum incubator set to 25 Hg, 37° C.+2° C. for 2-3 hours. A process for preparing plates as herein describes is depicted in FIG. 1.

### Example 3: Using Plates to Make an ELISA

An indirect enzyme-linked immunosorbent assay (ELISA) was designed using the Bartonella henselae antigen coated plates containing the test 17 kDa antigen.

Discarded human clinical serum samples were obtained from a clinical laboratory after all assays had been performed, the associated results analyzed and reported, and the appropriate retention time had expired. The samples were first screened for the presence of antibodies to Bartonella henselae by performing an immunofluorescent assay (IFA). All samples were run on the Euroimmun Anti-Bartonella henselae IIFT (IgG) according to the manufacturer's instructions.

The kit is suitable for diagnosing Bartonella henselae infections. But the method was not quantitative, negative results did not exclude infection as antibodies may have not been present in detectable quantities, and cross reactivity may be noted with other species such as: Bartonella quintana, Bartonella bacilliformis, Bartonella clarridgeiae and Afipiafelis (Source: Euroimmun IFU). In total, 70 samples were screened and the results recorded.

After screening by IFA, the serum samples were diluted 1:100 in 1% BSA dilution buffer and vortexed rapidly. 100 μL of the diluted samples were added to the wells of the microtiter plate coated with the test 17 kDa antigen. The plate was coated with an adhesive foil seal and allowed to incubate at room-temperature (20° C.+5° C.) for 1 hour.

After the incubation, the wells of the microtiter plate were washed 5× with a volume of 300 μL 1×PBS/0.05% Tween per well, utilizing a 60-second residence time between each wash.

Goat anti-human IgG (γ-chain) HRP conjugated detection antibody was prepared by first rehydrating with 1 mL molecular-grade H₂O and diluting to 1:10,000 in 1% BSA dilution buffer for a working concentration of 0.01 mg/mL.

After washing, the plate was tapped onto a paper towel to absorb any excess liquid prior to adding 100 μL diluted detection antibody per well. The plates were coated with an adhesive foil seal and allowed to incubate at room-temperature (20° C.+5° C.) for 30 minutes. After the incubation, the wells of the microtiter plate were washed 5× with a volume of 300 μL 1×PBS/0.05% Tween per well, utilizing a 60-second residence time between each wash.

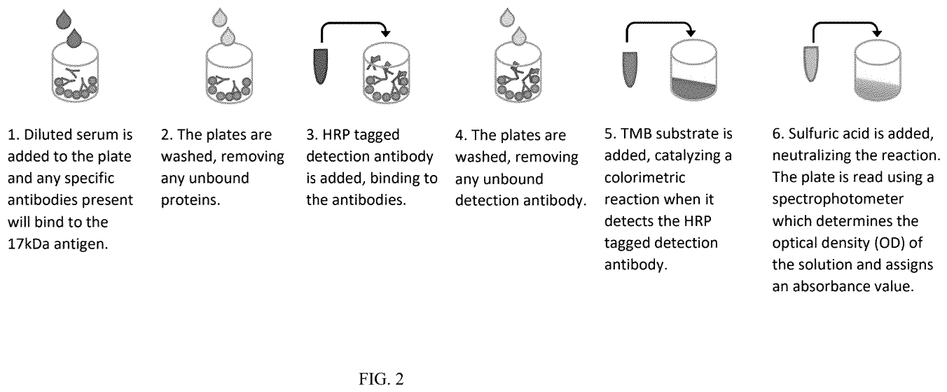

After washing, the plate was tapped onto a paper towel to absorb any excess liquid prior to adding 100 μL 3,3′,5,5′-Tetramethylbenzidine (TMB) substrate to each of the wells. The plate was coated with an adhesive foil seal and allowed to incubate at room-temperature (20° C.+5° C.) for 10 minutes. After the 10 minutes had expired, 100 μL 2N sulfuric acid (H₂SO₄) solution in H₂O was added to each of the wells to neutralize the reaction An example of the method as described herein is depicted in FIG. 2.

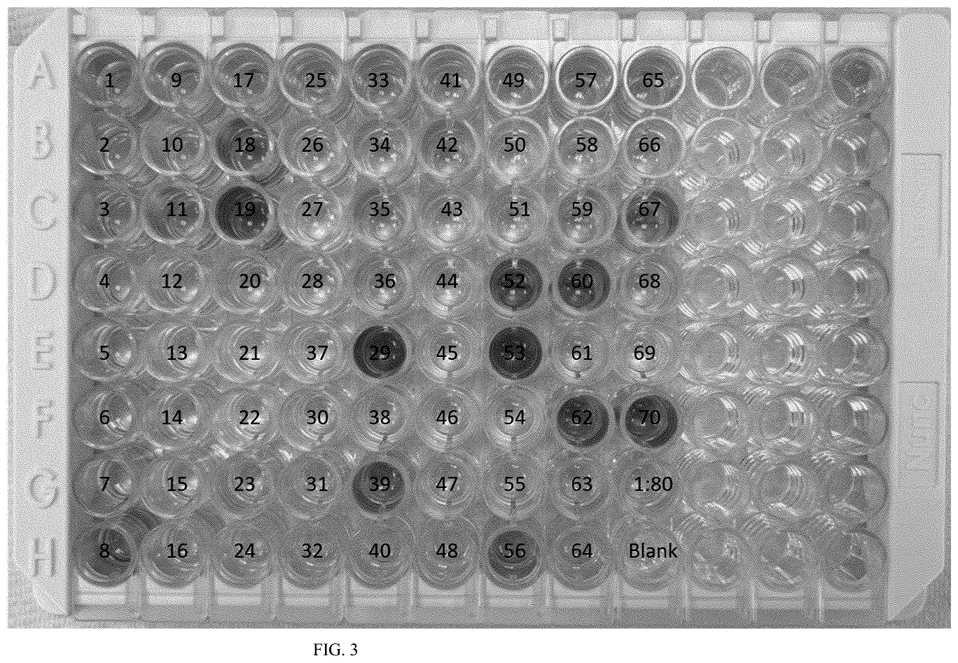

The absorbance values of the plate were read using a spectrophotometer set to an optical density (OD) of 450 nm, no reference wavelength. An example of an ELIZA plate with the 70 samples is depicted in FIG. 3. The intensity of the absorbance values directly correlates to the detection of the level of antibodies to Bartonella henselae.

When results of the IFA and the ELISA were compared, a clear negative cutoff was established at an absorbance value of 0.220 and a clear positive cutoff at an absorbance value of 1.000.57 of the 70 IFA screened samples were concordant when run on the ELISA, providing a concordance rate of 81.4%. 13 of the 70 samples were positive when the above cutoffs were used, and 7 samples were negative providing a positivity rate of 18.6%. The remaining 50 samples were scored as “negative/equivocal.”

Without calibrator controls, the ELISA was unable to accurately determine where the positive, negative, and equivocal thresholds lie except in cases of extreme positivity or negativity.

The results provided a means by which to detect the presence of antibodies to Bartonella henselae. To measure the concentration of the antibodies or to detect B. henselae infection, the standards and calibrator controls were developed as disclosed herein and incorporated into the ELISA.

### Example 4: Using the Antigen to Purify the Standard from the Human Serum

Antibodies were isolated from sera, and anti-17 kDa antigen antibodies affinity purified from the isolated antibodies, as follows. All sera determined to be highly reactive to Bartonella henselae by the IFA screening in the previous step were pooled. 4 mg of the 17 kDa target antigen was dialyzed against 4 L of 100 mM 3-(N-morpholino)propanesulfonic acid (MOPS) buffer, pH 7.5 overnight in a Thermo-Scientific™ 7K MWCO Slide-a-Lyzer™ cassette to conduct a buffer exchange prior to chromatography.

The pooled sera were purified utilizing a 5 mL Cytiva® HiTrap Protein G HP column to isolate the pure non-specific immunoglobulins. 5 mL of the pooled positive serum was centrifuged at 4° C., 1,300 rpm, for 10 minutes. The centrifuged serum was combined 1:1 with binding buffer containing 20 mM Sodium phosphate, pH 7.0. The Cytiva® HiTrap Protein G HP column was flushed with 50 mL binding buffer containing 20 mM Sodium phosphate, pH 7.0 at a rate of 5 mL/minute to clean the column of any storage buffers and alcohols.

The serum and binding buffer mixture was pushed through the Cytiva® HiTrap Protein G HP column at a rate of 1 mL minute. The Cytiva® HiTrap Protein G HP column was flushed with 50 mL binding buffer containing 20 mM Sodium phosphate, pH 7.0 at a rate of 5 mL/minute to clean the column of any unbound proteins, albumin, and excess serum. 24×1.5 mL Eppendor™ tubes were set up on a rack and 200 μL 1M Tris pH 8.0 was added to each one. 24×1.2 mL fractions of the purified immunoglobulin was eluted using 0.1M Glycine-HCl pH 2.7 elution buffer.

A small portion of each fraction was run on SDS-PAGE utilizing a Bio-Rad® 4-12% Criterion™ XT Bis-Tris protein gel and the Bio-Rad® XT MES Buffer Kit to analyze the location of the eluted immunoglobulin in the fractions. The most positive fractions, 6-21, were combined, diluted 1:1 with 1×PBS, and set aside. Approximately 2 mL Affi-Gel© 10 was added to a 15 mL conical tube and allowed to settle to obtain a 1 mL gel bed.

The supernatant was decanted from the gel bed and the gel suspended in 10 mL cold molecular grade H₂O. The gel was centrifuged at 4° C., 1,000×G for 30 seconds. This process was repeated and the gel was washed one more time with 10 mL cold molecular grade H₂O. The supernatant was decanted and the washed gel bed re-suspended in 1 mL cold 100 mM MOPS, pH 7.5.4 mg of the dialyzed target antigen was combined with the 1 mL gel bed and rocked on a platform overnight at 4° C. to allow the antigen to bind to the immunoaffinity support.

After rocking overnight, the affinity gel was removed from the rocker and poured into a 1.5×5 cm Bio-Rad® Econo-Column containing a stopcock and tubing. The column was washed once with 10 mL 1×PBS pH 7.2 at a rate of 1 drop every 3-5 seconds. This procedure was repeated with 10 mL elution buffer containing 100 mM glycine HCl/150 mM NaCl pH 2.4. The washing procedure was repeated one last time with 10 mL 1×PBS pH 7.2. The diluted fraction/PBS mixture was applied to the column and allowed to flow through at a rate of 1 drop every 3-5 seconds or 1-2 mL per minute.

The flow-through was re-applied 4× for a total of 5 passes. 12×1.5 mL Eppendorf™ tubes were set up on a rack and 200 μL 1M Tris pH 8.0 was added to each one. 12×1.2 mL fractions of the specific immunoglobulin standard were eluted using 100 mM glycine HCl/150 mM NaCl pH 2.4 elution buffer.

A small portion of each fraction was run on SDS-PAGE to analyze where the standard eluted in the liquid fractions. The results of the two-step purification method were quantified utilizing the Invitrogen IgG (Total) Human ELISA kit according to the manufacturer's instructions. The standard was diluted to 1,000 ng/mL and excess aliquots were flash-frozen in a dry-ice/EtOH bath. In total, 4.8 mL of the bioidentical immunoglobulin standard was obtained at a concentration of 16,559.09 ng/mL. The final yield was 79,483.7 ng.

This method was also used to effectively purify specific immunoglobulin M (IgM) from human serum. The Cytiva© HiTrap Protein G HP column and the Invitrogen IgG (Total) Human ELISA kit was replaced with the Cytiva© HiTrap IgM HP column and the Invitrogen IgM Human ELISA kit. The final yield was 559,458.48 ng over 4.8 mL from 5 mL serum. In an example (not shown), affinity purified target anti-17 kDa antigen IgM antibodies could be included in a calibration sample, such as when anti-17 kDa antigen IgM antibodies are isolated from a sample for use in an immunoassay with one or more calibration sample including anti-17 kDa antigen IgM antibodies affinity purified using a target 17 kDa antigen.

### Example 5: Using a Base Calibration Sample to Calibrator Samples Such as for Use in an Immunoassay

The pooled sample of affinity purified anti-17 kDa antigen antibodies was diluted further to 100 ng/mL and run on the designed ELISA in order to assess reactivity and to determine the linearity of a final product. Healthy donor serum was obtained and diluted 1:100 into dilution buffer containing 1% BSA. This achieved a standard dilution buffer containing negative human background serum at a dilution factor of 1:100. Calibration sample was diluted to 100 ng/mL in said standard dilution buffer. 1:1 serial dilutions were made into the standard dilution buffer to achieve an eight fold dilution series from 100 ng/mL down to 0.78 ng/mL.

These standards were added to the designed ELISA during the sample addition step. After obtaining the absorbance values via spectrophotometry, a standard curve was generated by plotting the concentration on the X axis against the absorbance value on the Y axis. The resulting R² value was 0.9964 between 25 ng/mL and 0.78 ng/mL.

### Example 6: Adding Calibrator Samples to an ELISA

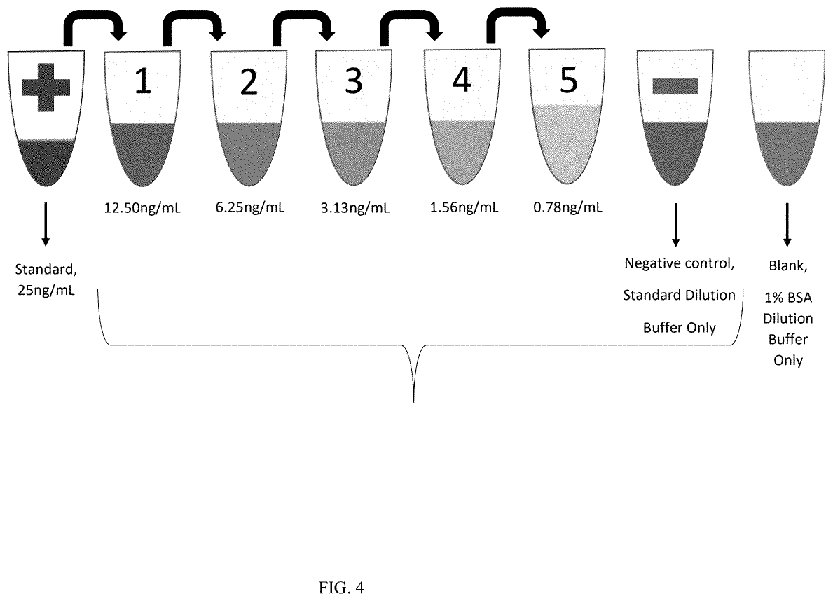

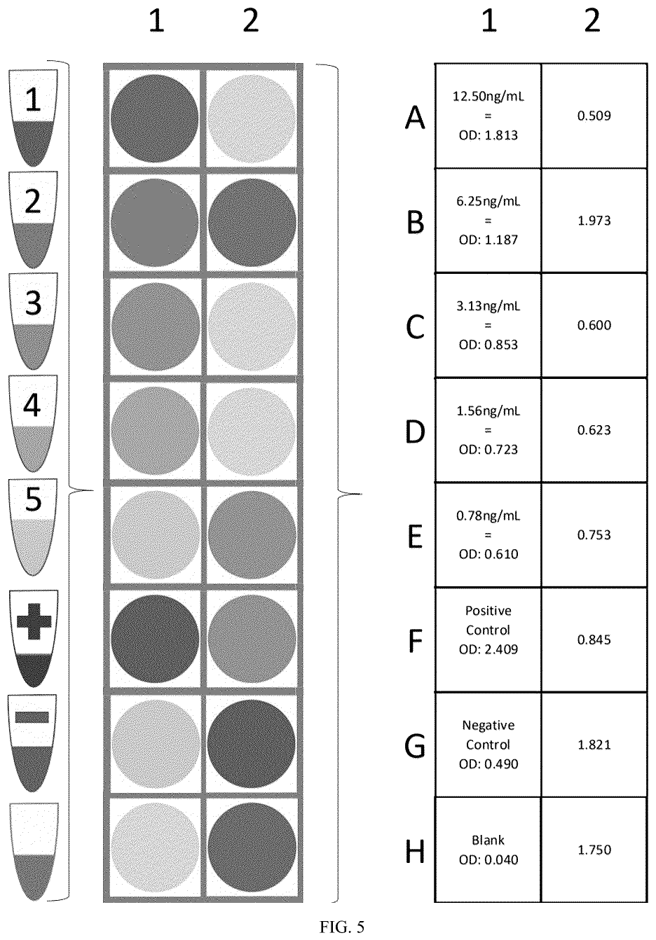

A range between 25 ng/mL and 0.78 ng/mL was determined by the previous experiments to be linear and these standards were run again on an ELISA as a set of 5 calibrator samples containing the concentrations of 12.5 ng/mL, 6.25 ng/mL, 3.13 ng/mL, 1.56 ng/mL, and 0.78 ng/mL, as depicted in FIG. 4. The 25 ng/mL standard was run as a positive control and the standard dilution buffer was run as a negative control containing 0.00 ng/mL of the bioidentical specific immunoglobulin standard, as depicted in FIG. 5. The panel of 70 IFA confirmed sera were run with these calibrator samples and controls. The samples were compared to the results of the standard curve. When using the 12.5 ng/mL standard as a positive cutoff, the same 13 samples were deemed to be positive, and 13 samples disagreed with the IFA results across both runs, highlighting the inherent drawbacks to the IFA method.

Identical concordance between samples on both runs in conjunction with the identical positive samples indicates that the calibration samples successfully compensate for any variables such as instrument readings caused by differential environmental conditions, non-specific background, or multiple operators.

### Example 7: Analyzing and Interpreting the Results

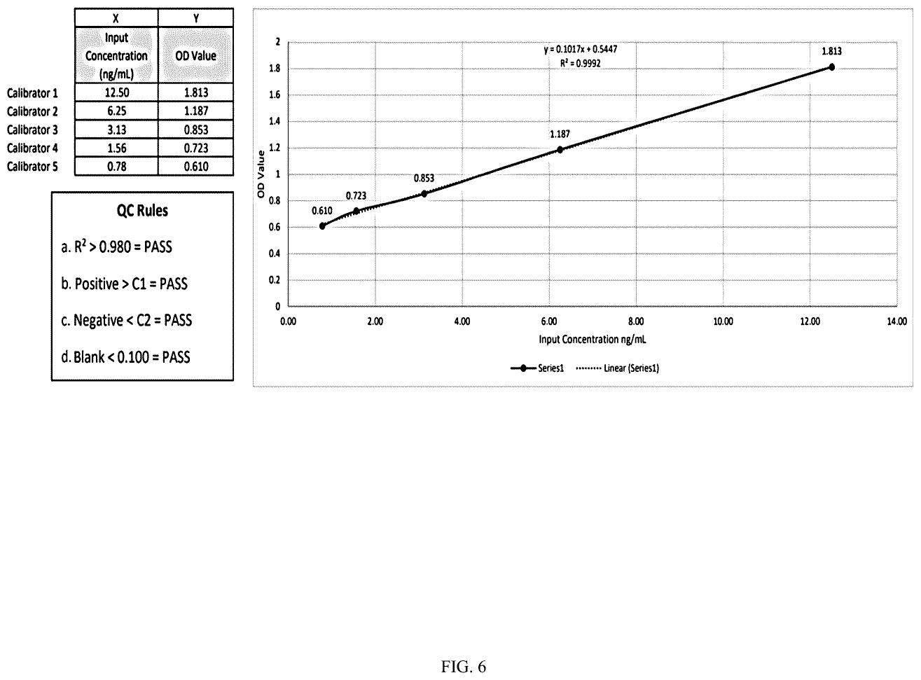

When using the 25 ng/mL calibration sample as a positive control, the standard dilution buffer as a negative control, 1% BSA dilution buffer as a blank and 12.5 ng/mL-0.78 ng/mL calibration samples, thresholds were easily able to be determined. The standards were highly linear with an R² value of 0.9992, as depicted in FIG. 6. A quality check of the data was performed to ensure reliability of results produced by this method.

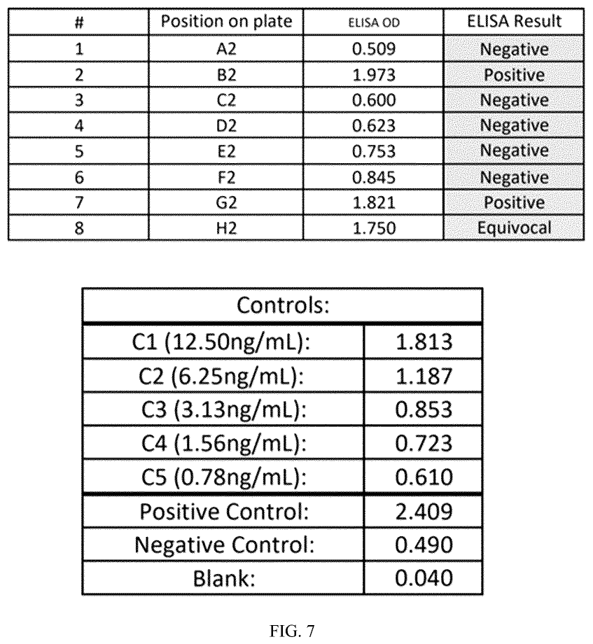

An example of results from an ELISA performed as described herein is depicted in FIG. 7. A sample with an absorbance value equal to or above that generated by the 12.5 ng/mL calibration sample was determined to be positive for anti-17 kDa antigen antibodies. A sample with an absorbance value equal to or below the 6.25 ng/mL calibration sample is determined to be negative for anti-17 kDa antigen antibodies. A sample with an absorbance value between these two levels was determined to be equivocal, with re-testing recommended. and the sample should be re-tested before reporting any results. See Table 2.

TABLE 2: Results for determining positivity or negativity of a test sample for anti-17 kDa antigen antibodies.

| Concentration | Result | Interpretation |
|---|---|---|
| ≥12.5 ng/ml | Positive | Positive specimen; reactive |
| | | for IgG antibodies indicating |
| | | previous exposure to B. |
| | | henselae or a |
| | | previous/current infection. |
| >6.25 nh/ml and <12.5 | Equivocal | Specimen considered for |
| ng/ml | | retesting |
| ≤6.25 ng/ml | Negative | No detectable anti-17 kDa |
| | | antibodies in sample |

When the 70 sample IFA concordance panel was analyzed in this manner, it had an 81.4% concordance rate with the IFA. The positivity rate for the panel of 70 samples was 18.6% with 13 samples positive, 7 equivocal, and 50 negative.

Although some non-limiting examples have been depicted and described in detail herein, it will be apparent to those skilled in the relevant art that various modifications, additions, substitutions, and the like can be made without departing from the spirit of the present disclosure and these are therefore considered to be within the scope of the present disclosure as defined in the claims that follow.

It should be appreciated that all combinations of the foregoing concepts and additional concepts discussed in greater detail herein (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein and may be used to achieve the benefits and advantages described herein.

What is claimed is:

1. A method of preparing a calibration sample for detection of anti-Bartonella henselae antibodies in a subject, comprising
obtaining one or more control sample from one or more human control subject infected with Bartonella henselae,
isolating antibodies from the one or more control sample to obtain a solution comprising isolated antibodies,
affinity purifying, from the isolated antibodies, antibodies that specifically bind target Bartonella henselae 17 kDa antigen or a fragment thereof to obtain a solution of purified antibodies, and
diluting the solution of purified antibodies to a predetermined protein concentration to obtain the calibration sample.

2. The method of claim 1, wherein the target Bartonella henselae 17 kDa antigen comprises a sequence of amino acids and the sequence is set out in SEQ ID NO: 1.

3. The method of claim 1, wherein the target Bartonella henselae 17 kDa antigen comprises a sequence of amino acids and the sequence is set out in SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13.

4. The method of claim 1, wherein the target Bartonella henselae 17 kDa antigen comprises a sequence of amino acids and the sequence is set out in SEQ ID NO: 13.

5. The method of claim 1, wherein the obtaining one or more control sample from one or more human control subject infected with Bartonella henselae comprises obtaining a serum sample from each of a plurality of human control subjects and pooling the serum samples.

6. The method of claim 1, further comprising freezing the calibration sample.

7. A method of detecting anti-Bartonella henselae antibodies in a test subject, comprising
contacting a peptide with a subject sample, comprising antibodies, from the test subject, wherein the peptide comprises a test Bartonella henselae 17 kDa antigen or a fragment thereof, and detecting a test signal indicating subject sample antibodies binding to the peptide;
contacting the peptide with a calibration sample, comprising antibodies, wherein the calibration sample comprises a threshold protein concentration, and detecting a calibration signal indicating calibration sample antibodies binding to the peptide, wherein
the calibration sample was prepared by:
obtaining one or more control sample from one or more human control subject infected with Bartonella henselae,
isolating antibodies from the one or more control sample to obtain a solution comprising isolated antibodies,
affinity purifying, from the isolated antibodies, antibodies that specifically bind a target Bartonella henselae 17 kDa antigen or a fragment thereof to obtain a solution of purified antibodies,
diluting the solution of purified antibodies to a predetermined protein concentration to obtain a base calibration sample, and
diluting the base calibration sample to obtain the calibration sample having a threshold protein concentration; wherein
detecting anti-Bartonella henselae antibodies in the test subject comprises detecting a test signal that equals or exceeds the calibration signal.

8. The method of claim 7, wherein the target Bartonella henselae 17 kDa antigen comprises a sequence of amino acids and the sequence is set out in SEQ ID NO: 1.

9. The method of claim 7, wherein the target Bartonella henselae 17 kDa antigen comprises a sequence of amino acids and the sequence is set out in SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13.

10. The method of claim 7, wherein the target Bartonella henselae 17 kDa antigen comprises a sequence of amino acids and the sequence is set out in SEQ ID NO: 13.

11. The method of claim 7, wherein the obtaining one or more control sample from one or more human control subject infected with Bartonella henselae comprises obtaining a serum sample from each of a plurality of human control subjects and pooling the serum samples.

12. The method of claim 7, wherein the threshold protein concentration is about 12.5 ng/ml.

13. The method of claim 7, wherein the detecting the test signal and the detecting the calibration signal comprise performing an enzyme-linked immunosorbent assay (ELISA).

14. The method of claim 7, wherein the target Bartonella henselae 17 kDa antigen or a fragment thereof comprises the test Bartonella henselae 17 kDa antigen or a fragment thereof.

15. A kit, comprising
a test Bartonella henselae 17 kDa antigen or a fragment thereof and a calibration sample, wherein the calibration sample comprises antibodies that bind a target Bartonella henselae 17 kDa antigen or a fragment thereof; wherein
the calibration sample was prepared by:
obtaining one or more control sample from one or more human control subject infected with Bartonella henselae,
isolating antibodies from the one or more control sample to obtain a solution comprising isolated antibodies,
affinity purifying, from the isolated antibodies, antibodies that specifically bind the target Bartonella henselae 17 kDa antigen or a fragment thereof to obtain a solution of purified antibodies, and
diluting the solution of purified antibodies to a predetermined protein concentration to obtain the calibration control sample.

16. The kit of claim 15, wherein the target Bartonella henselae 17 kDa antigen comprises a sequence of amino acids and the sequence is set out in SEQ ID NO: 1.

17. The kit of claim 15, wherein the target Bartonella henselae 17 kDa antigen comprises a sequence of amino acids and the sequence is set out in SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13.

18. The kit of claim 15, wherein the target Bartonella henselae 17 kDa antigen comprises a sequence of amino acids and the sequence is set out in SEQ ID NO: 13.

19. The kit of claim 15, wherein the obtaining one or more control sample from one or more human control subject infected with Bartonella henselae comprises obtaining a serum sample from each of a plurality of human control subjects and pooling the serum samples.

20. The kit of claim 15, wherein the test Bartonella henselae 17 kDa antigen or a fragment thereof is attached to a substrate.

21. The kit of claim 15, wherein the target Bartonella henselae 17 kDa antigen or a fragment thereof comprises the test Bartonella henselae 17 kDa antigen or a fragment thereof.

22. A method of treating a human subject for Bartonella henselae infection, comprising
selecting the subject and administering an antibiotic to the subject, wherein selecting the subject comprises obtaining or having obtained a test for detecting anti-Bartonella henselae antibodies in the subject, wherein the test comprises:
contacting a peptide with a subject sample, comprising antibodies, from the subject, wherein the peptide comprises a test Bartonella henselae 17 kDa antigen or a fragment thereof, and detecting a test signal indicating subject sample antibodies binding to the peptide;
contacting the peptide with a calibration sample, comprising antibodies and a protein concentration of about 12 ng/ml, and detecting a calibration signal indicating calibration sample antibodies binding to the peptide, wherein
the calibration sample was prepared by
obtaining one or more control sample from one or more human control subject infected with Bartonella henselae,
isolating antibodies from the one or more control sample to obtain a solution comprising isolated antibodies,
affinity purifying, from the isolated antibodies, antibodies that specifically bind a target Bartonella henselae 17 kDa antigen or a fragment thereof to obtain a solution of purified antibodies,
diluting the solution of purified antibodies to a predetermined protein concentration to obtain a base calibration sample, and
diluting the base calibration sample to obtain the calibration sample, and the test signal equals or exceeds the calibration signal.

23. The method of claim 22, wherein the antibiotic comprises one or more of a macrolide, a tetracycline, and an aminoglycoside.

24. The method of claim 22, wherein the antibiotic is selected from the group consisting of azithromycin, erythromycin, doxycycline, gentamicin, and any combination of two or more of the foregoing.

25. The method of claim 22, wherein the antibiotic is selected from the group consisting of azithromycin, erythromycin and doxycycline, and gentamicin and doxycycline.

26. The method of claim 22, wherein the target Bartonella henselae 17 kDa antigen comprises a sequence of amino acids and the sequence is set out in SEQ ID NO: 1.

27. The method of claim 22, wherein the target Bartonella henselae 17 kDa antigen comprises a sequence of amino acids and the sequence is set out in SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13.

28. The method of claim 22, wherein the target Bartonella henselae 17 kDa antigen comprises a sequence of amino acids and the sequence is set out in SEQ ID NO: 13.

29. The method of claim 22, wherein the obtaining one or more control sample from one or more human control subject infected with Bartonella henselae comprises obtaining a serum sample from each of a plurality of human control subjects and pooling the serum samples.

30. The method of claim 22, wherein the detecting the test signal and the detesting the calibration control signal comprise performing an enzyme-linked immunosorbent assay (ELISA).

31. The method of claim 22, wherein the target Bartonella henselae 17 kDa antigen or a fragment thereof comprises the test Bartonella henselae 17 kDa antigen or a fragment thereof.


SEQUENCE LISTING (13 sequences)

SEQ ID NO: 1 - protein, 148 aa, synthetic construct
MKKYSLVTLLSLFCISHAKAQTATLTDEYYKKALENTQKLDVAKSQTAESIYESATQTANKIKDINNQLANLKADTKTKPEQLQALQIELTLLQAQLQADTLKIQSLAMIQAKDTKTKEELREEQTQKKHEDLQKQLKEKLEKSDVQL

SEQ ID NO: 2 - protein, 72 aa, synthetic construct
KTKPEQLQALQIELTLLQAQLQADTLKIQSLAMIQAKDTKTKEELREEQTQKKHEDLQKQLKEKLEKSDVQL

SEQ ID NO: 3 - protein, 72 aa, synthetic construct
KTKPEQLQALQIELTLLQAQLQADTLKIQSLAMIQAKDTKTKEELREEQTQKKHEDLQKQLKEKLEKSDVRL

SEQ ID NO: 4 - protein, 72 aa, synthetic construct
KTKPEQLQALQIDLTLLQAQLQADTLKIQSLAMIQAKDTKTKEELREEQTQKKHEDLQKQLKEKLEKSDVQL

SEQ ID NO: 5 - protein, 72 aa, synthetic construct
KTKPEQLQALQIELTVLQAQLQADTLKIQSLAMIQAKDTKTKEELREEQTQKKHEDLQKQLKEKLEKSDVQL

SEQ ID NO: 6 - protein, 71 aa, synthetic construct
KTKPEQLQALIELTLLQAQLQADTLKIQSLAMIQAKDTKTKEELREEQTQKKHEDLQKQLKEKLEKSDVQL

SEQ ID NO: 7 - protein, 73 aa, synthetic construct
KTKPEQLQALQIELTLLVQAQLQADTLKIQSLAMIQAKDTKTKEELREEQTQKKHEDLQKQLKEKLEKSDVQL

SEQ ID NO: 8 - protein, 18 aa, synthetic construct
EDLQKQLKEKLEKSDVRL

SEQ ID NO: 9 - protein, 18 aa, synthetic construct
DDLQKQLKEKLEKSDVRL

SEQ ID NO: 10 - protein, 18 aa, synthetic construct
EDIQKQLKEKLEKSDVRL

SEQ ID NO: 11 - protein, 18 aa, synthetic construct
EDLQKQVKEKLEKSDVRL

SEQ ID NO: 12 - protein, 18 aa, synthetic construct
LKQDEQKLEKLEKSDVRL

SEQ ID NO: 13 - protein, 71 aa, synthetic construct
TKPEQLQALQIELTLLQAQLQADTLKIQSLAMIQAKDTKTKEELREEQTQKKHEDLQKQLKEKLEKSDVQL